(19) United States
(12) Patent Application Publication
(10) Pub. No.: US 2023/0329958 A1
(43) Pub. Date: Oct. 19, 2023

(54) Massage head assembly

(71) Applicant: XIAMEN COMFIER TECHNOLOGY CO., LTD., Fujian (CN)

(72) Inventors: Jianqing LI, Fujian (CN); Song YANG, Fujian (CN); Zhicheng ZHANG, Fujian (CN)

(21) Appl. No.: 18/028,756

(22) PCT Filed: Sep. 15, 2021
(86) PCT No.: PCT/CN2021/118433

§ 371 Date: Mar. 28, 2023

(30) Foreign Application Priority Data
Nov. 19, 2020 (CN) 202022691878.8

(51) Int. Cl. A61H 7/00

(52) U.S. Cl. CPC A61H 7/007; A61H 2201/0107; A61H 2201/0207; A61H 2201/0221; A61H 2201/169

(57) ABSTRACT

A massage head assembly, including a massage head base disc (1), a massage head support (2), and silicone massage heads (3). The silicone massage heads (3) are fixed on the massage head support (2), the massage head support (2) is detachably fixed on the massage head base disc (1), the silicone massage heads (3) are hollow, and a buffer space (31) is formed between inner walls of the silicone massage heads (3) and the massage head support (2). The overall structure of the massage head assembly is simple, and when used for massaging the human body, the massage head assembly provides a comfortable massage.

### FIELD OF THE INVENTION

The present invention relates to the technical field of massage apparatus and more particularly pertains to a massage head assembly.

### BACKGROUND OF THE INVENTION

Along with social development, there is a rapid growth of the number of brainworkers who are mostly lack of exercise and entertainment, and thus suffering from bad mood and worsening health conditions over time. Many massagers are available on the market for mood relief and body relaxation. Stimulation with a massager helps one's body to relax and provides health supporting effects.

A massager in the prior art usually comprises a massager body and a massage head assembly disposed on the massager body; the massage head assembly comprises a massage head base and a massage head disposed on the massage head base; the massage head is made of plastic materials, which provides the massage head with an overly hard texture, and leads to an uncomfortable massage experience.

### BRIEF SUMMARY OF THE INVENTION

The present invention provides a massage head assembly with a simple structure, which is capable of providing a comfortable massage for users.

To attain the above object, the technical solutions of the present invention are as follows:

A massage head assembly, comprising a massage head base, a massage head holder and a silicone massage head; the silicone massage head is fixedly disposed on the massage head holder; the massage head holder is detachably disposed on the massage head base; the silicone massage head is of a hollow structure; the silicone massage head has an inner wall, and a buffer space is formed between the inner wall and the massage head holder.

An outer surface of the silicone massage head is covered with a cloth cover.

The massage head assembly further comprises an insert; the insert is formed between a bottom part of the silicone massage head and a top surface of the massage head holder by means of secondary plastic over-molding.

A plurality of positioning columns protrude from a bottom surface of the insert at intervals; a plurality of positioning holes are provided on the massage head holder; each of the positioning columns passes through the corresponding positioning hole.

A fitting portion protrudes upwards from a middle part of the massage head holder; the insert is engaged with the fitting portion.

The massage head assembly further comprises a heating member; the heating member is fixedly disposed inside the silicone massage head.

The massage head assembly further comprises a light board with a plurality of light beads; the heating member is fixedly disposed on the light board; the light board is fixedly disposed on the massage head holder; the massage head holder is provided with a wire outlet for a light board electrical connection wire of the light board and a heating member electrical connection wire of the heating member to pass through.

An accommodating column protrudes downwards from a middle part of the massage head holder; the massage head base is provided with a connection column; the connection column snaps into the accommodating column.

At least two positioning grooves recess into an inner wall of the accommodating column at intervals; at least two positioning rods protrude from an outer wall of the connection column at intervals; each of the positioning rods is slidably fitted inside the corresponding positioning groove.

A middle part of the massage head base is provided with an eccentric component.

By adopting the aforementioned technical solutions, the structure and design of the silicone massage heads provide the present invention with a simple overall structure; due to the good flexibility of the silicone massage head, and the buffer space formed between the inner wall of the silicone massage head and the massage head holder being capable of elastic deformation, the elasticity of the silicone massage head is increased, so that when the present invention is used to massage a human body, the silicone massage head can elastically deform to a larger degree, thereby providing a comfortable massage.

### BRIEF DESCRIPTION OF THE DRAWINGS

FIG. 1 shows a perspective view of the present invention;

FIG. 2 shows a partially exploded view of the present invention;

FIG. 3 shows another partially exploded view of the present invention.

As illustrated in the figures: 1 denotes the massage head base; 11 denotes the connection columns; 111 denotes the positioning rods; 12 denotes the eccentric component; 2 denotes the massage head holders; 21 denotes the positioning holes; 22 denotes the fitting portions; 23 denotes the wire outlets; 24 denotes the accommodating columns; 241 denotes the positioning grooves; 3 denotes the silicone massage heads; 31 denotes the buffer spaces; 4 denotes the cloth covers; 5 denotes the inserts; 51 denotes the positioning columns; 6 denotes the heating members; 61 denotes the heating member electrical connection wires; 7 denotes the light boards; 71 denotes the light beads; 72 denotes the light board electrical connection wires.

### DETAILED DESCRIPTION OF THE INVENTION

To attain the above objects and effects, the features and functions of the technical solutions and structure adopted by the present invention will be described in detail in the preferred embodiments of the present invention with reference to the accompanying drawings, so as to aid thorough understanding.

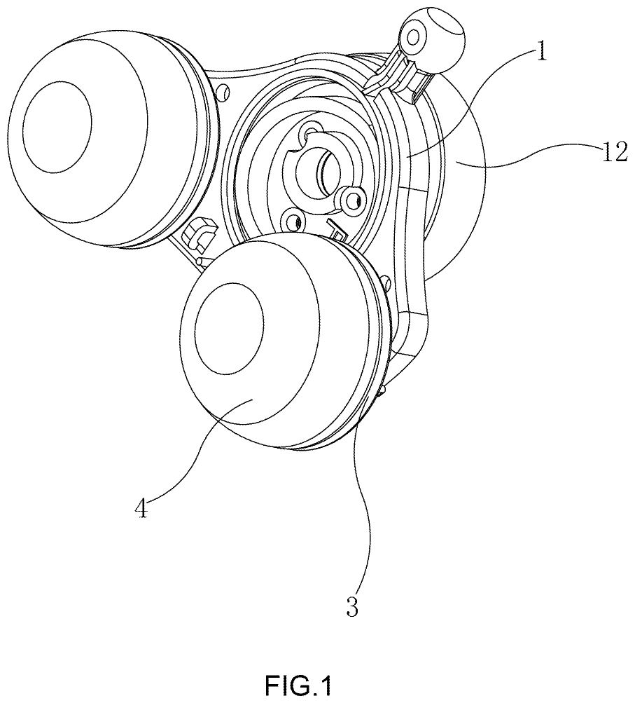

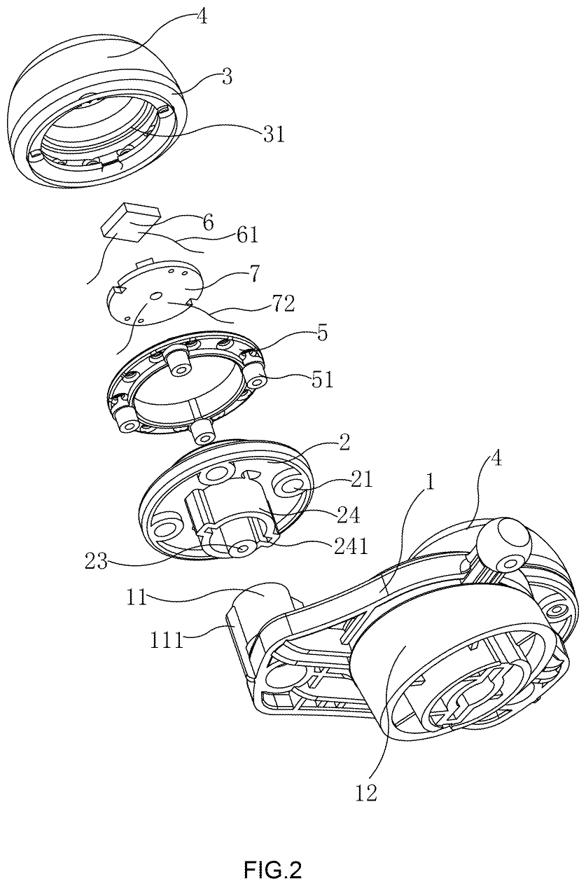

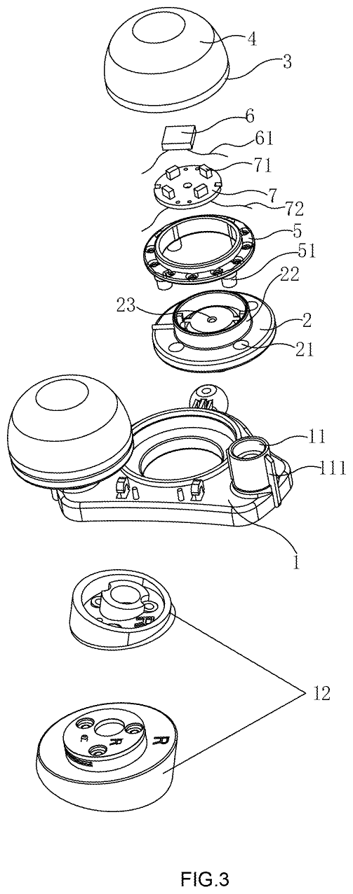

As illustrated in FIGS. 1-3, the present invention discloses a massage head assembly comprising a massage head base 1, massage head holders 2 and silicone massage heads 3; each of the silicone massage heads 3 is fixedly disposed on the corresponding massage head holder 2; each of the massage head holders 2 is detachably disposed on the massage head base 1; each of the silicone massage heads 3 is of a hollow structure; each of the silicone massage heads 3 has an inner wall, and a buffer space 31 is formed between the inner wall and the corresponding massage head holder 2.

Therefore, the structure and design of the silicone massage heads 3 provide the present invention with a simple overall structure; due to the good flexibility of the silicone massage heads 3, and the buffer space 31 formed between the inner wall of each of the silicone massage heads 3 and the corresponding massage head holder 2 being capable of elastic deformation, the elasticity of the silicone massage heads 3 is increased, so that when the present invention is used to massage a human body, the silicone massage heads 3 can elastically deform to a larger degree, thereby providing a comfortable massage.

Furthermore, an outer surface of each of the silicone massage heads 3 is covered with a cloth cover 4; each of the cloth covers 4 can be an abrasion resistant cloth; for instance, each of the cloth covers 4 can be, but is not limited to, a non-woven cloth or a woven cloth, which enables the present invention to perform a smooth and comfortable massage, and provides the silicone massage heads 3 with a better sliding effect.

Furthermore, the message head assembly of the present invention further comprises inserts 5 to facilitate fixed securement of the silicone massage heads 3 to the massage head holders 2; each of the inserts 5 is formed between a bottom part of each of the silicone massage heads 3 and a top surface of the corresponding massage head holder 2 by means of secondary plastic over-molding; the inserts 5 and the massage head holders 2 can be made of plastic materials.

Furthermore, a plurality of positioning columns 51 protrude from a bottom surface of each of the inserts 5 at intervals; a plurality of positioning holes 21 are provided on each of the massage head holders 2; each of the positioning columns 51 passes through the corresponding positioning hole 21 to facilitate positioning of the inserts 5 and the massage head holders 2 and forming of each of the inserts 5 between the bottom part of each of the silicone massage heads 3 and the top surface of the corresponding massage head holder 2 by means of secondary plastic over-molding.

Furthermore, a fitting portion 22 protrudes upwards from a middle part of each of the massage head holders 2; each of the inserts 5 is engaged with the corresponding fitting portion 22, so as to strengthen the connection between the inserts 5 and the massage head holders 2.

The inserts 5 can also be formed individually by injection molding; each of the cloth covers 4 covers an outer surface of the corresponding silicone massage head 3 to fix the corresponding insert 5 to the corresponding massage head holder 2; each of the silicone massage heads 3 is disposed on the corresponding insert 5 for placing into a silicone mold together to perform secondary plastic over-molding, so that each of the inserts 5 is fixedly disposed between the bottom part of the corresponding silicone massage head 3 and the top surface of the corresponding massage head holder 2.

Furthermore, to improve the massage effect of the present invention, the massage head assembly of the present invention further comprises heating members 6; each of the heating members 6 is fixedly disposed inside the corresponding silicone massage head 3; each of the heating members 6 can be formed inside the corresponding silicone massage head 3 by means of secondary plastic over-molding, or mounted independently inside the corresponding silicone massage head 3.

Furthermore, the massage head assembly of the present invention comprises light boards 7 each with a plurality of light beads 71; each of the heating members 6 is fixedly disposed on the corresponding light board 7; each of the light boards 7 is fixedly disposed on the corresponding massage head holder 2; each of the massage head holders 2 is provided with a wire outlet 23 for a light board electrical connection wire 72 of the corresponding light board 7 and a heating member electrical connection wire 61 of the corresponding heating member 6 to pass through; the light beads 71 are used to indicate the usage status of the present invention to users.

To facilitate assembly of the present invention, an accommodating column 24 protrudes downwards from the middle part of each of the massage head holders 2; the massage head base 1 is provided with connection columns 11; each of the connection columns 11 snaps into the corresponding accommodating column 24; at least two positioning grooves 241 recess into an inner wall of each of the accommodating columns 24 at intervals; at least two positioning rods 111 protrude from an outer wall of each of the connection columns 11 at intervals; each of the positioning rods 111 is slidably fitted inside the corresponding positioning groove 241, so as to facilitate fixed securement of the massage head holders 2 on the massage head base 1.

Furthermore, to improve the massage effect of the present invention, a middle part of the massage head base 1 is provided with an eccentric component 12, which enables the present invention to perform eccentric motions; in the present embodiment, the numbers of the positioning grooves 241 and the positioning rods 111 are both two; the numbers of the silicone massage heads 3, the massage head holders 2, the inserts 5, the light boards 7 and the heating members 6 are all two; the silicone massage heads 3 are disposed on two sides of the eccentric component 12 at intervals; the numbers of the connection columns 11 and the accommodating columns 24 are both two.

The technical solutions and features of the present invention are disclosed above; the numbers of the components of the present invention shall not be limited to the above; those skilled in the art may make equivalent changes and modifications without departing from the essence of the present invention; therefore, it should be understood that the protection scope of the present invention shall not be limited to the embodiments disclosed herein; equivalent changes and modifications without departing from the essence of the present invention and covered by the claims shall also fall within the protection scope of the present invention.

What is claimed is:

1. A massage head assembly, comprising a massage head base, a massage head holder and a silicone massage head; the silicone massage head is fixedly disposed on the massage head holder; the massage head holder is detachably disposed on the massage head base; the silicone massage head is of a hollow structure; the silicone massage head has an inner wall, and a buffer space is formed between the inner wall and the massage head holder.

2. The massage head assembly of claim 1, wherein an outer surface of the silicone massage head is covered with a cloth cover.

3. The massage head assembly of claim 1, further comprising an insert; the insert is formed between a bottom part of the silicone massage head and a top surface of the massage head holder by means of secondary plastic over-molding.

4. The massage head assembly of claim 3, wherein a plurality of positioning columns protrude from a bottom surface of the insert at intervals; a plurality of positioning holes are provided on the massage head holder; each of the positioning columns passes through the corresponding positioning hole.

5. The massage head assembly of claim 4, wherein a fitting portion protrudes upwards from a middle part of the massage head holder; the insert is engaged with the fitting portion.

6. The massage head assembly of claim 1, further comprising a heating member; the heating member is fixedly disposed inside the silicone massage head.

7. The massage head assembly of claim 6, further comprising a light board with a plurality of light beads; the heating member is fixedly disposed on the light board; the light board is fixedly disposed on the massage head holder; the massage head holder is provided with a wire outlet for a light board electrical connection wire of the light board and a heating member electrical connection wire of the heating member to pass through.

8. The massage head assembly of claim 1, wherein an accommodating column protrudes downwards from a middle part of the massage head holder; the massage head base is provided with a connection column; the connection column snaps into the accommodating column.

9. The massage head assembly of claim 8, wherein at least two positioning grooves recess into an inner wall of the accommodating column at intervals; at least two positioning rods protrude from an outer wall of the connection column at intervals; each of the positioning rods is slidably fitted inside the corresponding positioning groove.

10. The massage head assembly of claim 1, wherein a middle part of the massage head base is provided with an eccentric component.
